(19) United States
(12) Patent Application Publication
(10) Pub. No.: US 2021/0308350 A1
(43) Pub. Date: Oct. 7, 2021

(54) Device having an artificial gills system and use thereof for supporting a newborn

(71) Applicant: Michael TCHIRIKOV, Halle/Saale (DE)

(72) Inventor: Michael Tchirikov, Halle/Saale (DE)

(21) Appl. No.: 17/287,376

(22) PCT Filed: Oct. 25, 2019
(86) PCT No.: PCT/EP2019/079213

§ 371 Date: Apr. 21, 2021

(30) Foreign Application Priority Data
Oct. 25, 2018 (DE) 10 2018 126 634.0

(51) Int. Cl. A61M 1/32; A61M 1/16; A61G 11/00

(52) U.S. Cl. CPC A61M 1/32; A61M 1/1698; A61G 11/00; A61M 2210/1466; A61M 2202/049; A61M 2205/581; A61M 2205/583; A61M 2240/00

(57) ABSTRACT

A device and a method for supporting a human being, preferably a premature infant between the 21/0 and 28/0 week of pregnancy (SSW), The device comprises at least one through-flow system, comprising a number of fluid-permeable elements and connecting elements for the connection to an umbilical arterial catheter and an umbilical. venous catheter and a through-flow lumen for passing modified amniotic fluid enriched with oxygen or an oxygen-containing gas mixture through the fluid-permeable elements, and at least one connection for introducing the modified amniotic fluid into the through-flow system. The through-flow systema is configured in such a way that the modified amniotic fluid is guided through the fluid-permeable elements of the through-flow system while the fetal blood is passed along the outer side of the fluid-permeable elements, but directed past the through-flow lumen, whereby a gas exchange occurs.

### TECHNICAL AREA

The present invention relates to a device and a method for life support of a human being, preferably a newborn, in particular an extremely premature infant, between the 21/0 and 28/0 weeks of gestation (WG).

### STATE OF THE ART

The births of infants before the 24th week of gestation (WG) weighing less than 500 g, with no sign of life, are considered “late-term miscarriages” and are not statistically recorded as births. Worldwide, late-term miscarriage affects several million children in the developed countries. Compared to full-term babies, premature infants have a significantly higher risk of developing health complications immediately after birth or later in life. The earlier a baby is born, the more underdeveloped its organs are, and the higher the risk of developing health problems. Of all extremely premature infants (i.e. born before 28/0 WG), 40% die within the first five years (WHO report “Born too soon,” 2011). Furthermore, 91% of premature infants die in the 23rd week of gestation and 67% die in the 24th week of gestation (Stoll et al. JAMA 2015). Only rarely do extremely premature infants survive without serious late sequelae (Chen F. et al. Arch Dis Chld Fetal Neonatal 2016; 101:377-83). These include cerebral palsy, sensory and motor disabilities, learning and behavioral disorders, and often pulmonary problems. Only 6% of all premature infants born in the 22nd week of gestation survive to hospital discharge, with 95% to 96% of them showing significant physical and/or mental damage (Stoll et al. JAMA 2015). Born in the 22nd week of gestation, 89% of surviving premature infants suffer retinopathy with severity greater than 3 (relative blindness). In the 23rd week of gestation, 42% are affected. Only a percentage of less than 20% of premature infants survive to hospital discharge without the development of necrotizing enterocolitis, sepsis, meningitis, bronchiopulmonary hypoplasia, and/or marked cerebral hemorrhage in premature infants born before the 24th WG (Stoll et al. JAMA 2015). A similar picture, with sometimes drastically poor survival rates, is also provided by a recent study of neonates between 22 and 24 weeks of gestation (Noelle Younge et al., Survival and Neurodevelopmental Outcomes among Periviable Infants, The New England Journal of Medicine, Feb. 16, 2017, vol. 376, no. 7).

The lungs are among the most late developed organs in the fetus. This means that in case of extreme prematurity, the immature insufficient lungs may lead to short and/or long term health problems or death of the premature infant. The incidence of bronchopulmonary dysplasia is 72% in extremely premature infants<25/0 WG (Noelle Younge et al, Survival and Neurodevelopmental Outcomes among Periviable Infants, The New England Journal of Medicine, Feb. 16, 2017 vol. 376 no. 7). Pneumothorax often occurs when extremely immature lungs are ventilated. In most cases, the immature insufficient lungs are unable to provide normal sustained oxygen supply in the infant, resulting in hypoxic brain damage and/or pronounced cerebral hemorrhage.

To address this problem, methods and therapies using artificial lungs are being developed in order to increase the survival rate of extremely premature infants and prevent late complications.

In human fetuses, the placenta takes over the oxygen supply. Other important placental functions include transplacental active and/or passive transport of amino acids, fatty acids, microelements, vitamins, water, electrolytes, growth factors, hormones, cytokines and other regulatory substances (NO etc.). To some extent, substances such as amino acids, hormones, NO are also synthesized in the placenta itself. The disposal of fetal metabolites such as bilirubin or CO₂ takes place via the placenta. Afterwards, the maternal kidneys and lungs take over the disposal function.

Arterial blood is transported to the womb via the uterine vessels. The spiral arteries bring the blood across the basal plate of the placenta into the placental intervillous space. There, the exchange of O₂/CO₂ occurs between the maternal blood and the fetal blood. The fetal placental blood is located in the fetal capillaries of the placental villi and is separated from the maternal blood by a thin layer of tissue-syncytium and cytotrophoblast.

Under laboratory conditions, the O₂ binding curve of fetal hemoglobin is steeper than that of maternal blood. Due to the low physiological fetal pH, the Hb affinity is very similar to the adult Hb affinity under normal conditions. The oxygen binding curve is shifted to the right by the pH decrease. As a result of the pH decrease, hemoglobin releases oxygen more readily in fetal tissue. The CO₂- and H⁺-influence on the O₂-affinity of hemoglobin is also called the Bohr effect.

The dissociation of carbonic acid is promoted the more, the less the fetal hemoglobin is loaded with O₂ (Haldane effect). The normal fetal hemoglobin concentration increases from 10-12 g/dl in the 17th/18th WG to 14-15 g/dl at the expected date of birth.

Systems and artificial wombs are known to be used to increase the vital functions of a premature infant.

WO 2018/171905 A1 describes an artificial womb system for life support of newborns, in particular extremely premature infants between the 21/0 and 28/0 weeks of gestation, with an oxygenator and/or a gassing device for oxygen supply to the newborn or premature infant.

US 2014/0255253 A1 describes an artificial placenta oxygenating device for use with a premature infant. The device comprises a gas permeable membrane and a vascular network through which, for example, fluids containing nutrients can be supplied. Oxygen is supplied via an oxygenator through the umbilical cord using an umbilical catheter (cord catheter) that includes a venous catheter and an arterial catheter. A gas mixture containing 40% oxygen in nitrogen was administered via this catheter system.

Another way of oxygenating a premature baby is described in WO 2014/145494 A1, in which the circulatory system of the premature infant is coupled to an extracorporeal membrane that is part of an oxygen supply system. One embodiment provides that the oxygenator enriches the fetal blood with oxygen,

WO 2016/154319 A1 describes an artificial placenta in which microfluid channels are provided which are arranged between a membrane in such a way that fluid transport can take place through the membrane, for example to supply the fetus with nutrients. Regulation of gas exchange by pressure or amniotic fluid velocity along the membranes is not described. Furthermore, cell layers of at least two different cell types are provided, which adhere to the two sides of the membrane, The first cell type consists of, for example, primary human placental villous endothelial cells, while the second cell type comprises chorionic carcinoma cells.

Such membranes are also part of U.S. Pat. No. 4,556,489 A, which discloses a device for mass transfer through a membrane particularly suitable for use as a blood oxygenator.

Extracorporeal membrane oxygenation (ECMO) is often used to replace lung function. With the ECL (extracorporal life support) system, hemodynamic relief of the heart is also possible, because the circulation is supported. The blood is transported from the patient's venous system by means of a pump and, after passing through the oxygenator, is returned to the arterial system. The oxygen supply to the organism is composed of the ECMO/ECLS flow and the patient's remaining circulatory function. The connection to the extracorporeal circulation usually takes place via cannulas. The systems require blood heparinization to avoid thrombosis in the artificial system. The other problem is the destruction of erythrocytes which results in fetal anemia and increases bilirubin concentration.

The placenta systems described in the state of the art are inadequate in many respects, as they are unsuitable for the treatment of extremely premature infants.

Partridge et al. (An extra-uterine system to physiologically support the extreme premature lamb. Nature Communications, 2017; 8;15112) describe the use of an oxygenator (Quadrox-ID Pediatric oxygenator (Maquet Quadrox-ID Pediatric Oxygenator: Maquet Cardiopulmonary AG, Rastatt, Germany) and a Quadrox Neonatal Oxygenator (Maquet Quadrox-I Neonatal and Pediatric Oxygenator: Maquet Cardiopulmonary AG)) in a 2nd-3rd trimester fetal sheep model. The oxygenator is connected to the umbilical vessels via catheters. Out of a total of 33 fetuses, only 3 survived without complications for several weeks. Sepsis, heart failure, and hemorrhage were among the most common complications. To stabilize fetal blood gases in ranges PaO₂ 20-30 mm Hg and Pa CO₂ 35-45 mm Hg and to avoid excessive oxygen saturation, nitrogen had to be used to reduce the oxygen concentration of medical air from 21% to 11-14%. Oxidative stress, as an imbalance between free radicals and antioxidant defense mechanisms, is one of the main factors in the poor outcome of pregnancy (Sultana et al. 2017 Am J Reprod Immunol. 2017 May; 77(5)). Side effects may include hemolysis and embolism when using such oxygenators. In addition, post perfusion syndrome or organ damage may occur.

Modern oxygenators also need their own integrated system for blood warming. The formation of air bubbles is a well-known problem with oxygenators, which is why de-airing systems are often provided. Another problem is caused by the fact that the membranes of the oxygenator are dry on the side of the O₂ or gas mixture inflow, which significantly increases the risk of thrombus formation in the blood.

The alveolar space of the human lung is coated with surfactant. The surfactant consists mainly of phospholipids and is synthesized by type II pneumocytes and secreted into the alveoli to avoid surface tension. Between the capillary blood and the air are the alveolar epithelial cells and the endothelial cells. There is no direct contact between the blood and the air.

Upon contact with air, the coagulation system is activated. The activated clotting time (ACT) of the fetuses is increased to 150-180 s when using an oxygenator with heparin (10-400 UPS/h) to avoid thrombosis in the ECMO/ECL system.

Youngle et al. (New Engl J Med 2017) describes the rate of marked cerebral hemorrhage in extremely premature infants of 29%. Additional heparin infusions would significantly worsen this complication in human fetuses and premature infants. Also, the development of fetal anemia has been documented with the use of an oxygenator (Partridge et al 2017).

In summary, existing womb systems and oxygenators remain inadequate, especially with regard to efficient oxygen delivery in extremely premature infants. A system would be desirable that does not require blood heparinization to avoid thrombosis, destruction of erythrocytes, or fetal anemia.

### DESCRIPTION OF THE INVENTION

Against this background, it is the object of the present invention to provide an improved artificial system suitable for life support of a newborn, particularly an extremely premature infant between 21/0 and 28/0 weeks of gestation, and which avoids or at least reduces the disadvantages of the ECMO/ECL system in order to improve neonatal outcome.

This object is solved by a device and an ex-vivo method with the features of the following claims. Preferred embodiments can be found in the subclaims.

The core of the inventive device is an artificial flow-through system consisting of a number of fluid permeable elements, for example stacked or lamellar membranes or microporous material (e.g, tubes), enabling efficient O₂/CO₂ exchange in fetal blood, similar to a gill system in fish. It is provided that oxygen, which is dissolved in oxygenated amniotic fluid or present in an artificial womb chamber, is delivered to the fetal blood via the flow-through system according to the invention. The fetal blood is diverted from the blood vessels of the umbilical cord. In contrast to known systems, the system according to the invention oxygenates the amniotic fluid and not the fetal blood, The oxygenated amniotic fluid flows through the fluid-permeable elements, while the blood passes by on the outside. This facilitates gas exchange, similar to the gills in fish.

Instead of modified amniotic fluid, a plasma replacement solution can be used.

The flow-through system is preferably located in a container (i.e. housing or lumen) that also contains the fetus. This container serves as an artificial womb. In an alternative embodiment, the flow-through system is located outside the artificial womb in a separate housing. In a further embodiment, a container or housing can be dispensed with completely, i.e. the flow-through system itself is not integrated in any container.

The membranes or the microporous material of the flow-through systems can be connected either serially or in parallel in the direction of flow. The flow-through system is constructed like a gill system, i.e. it comprises a lumen through which the oxygenated amniotic fluid flows and a flow-through lumen via which blood is passed by.

The gill-like flow-through system enables efficient gas, electrolyte exchange and toxin and waste product disposal, e,g. of bilirubin, ammonia, nitrogen. Furthermore, osmoregulation via ion transport is possible.

The present invention is based on the idea that the fetus develops in the amniotic fluid. In this process, gas exchange takes place between the fetal blood and the modified amniotic fluid enriched with oxygen or an oxygen-containing gas mixture via the ultra-thin fetal skin, the mucous membranes and the fetal intestine of the fetus. The gas mixture preferably includes oxygen (O₂), carbon dioxide (CO₂), and/or nitrogen (N₂). Some organisms use a gill system for respiration, i.e. fish, crustaceans and mollusks, which supplies the organism with oxygen. In addition to respiratory functions, the gills also perform other functions, such as nitrogen excretion, osmoregulation, and food intake. For example, nitrogen is excreted through the gills in the form of ammonia. The lipid-soluble pollutants, which increasingly accumulate in higher concentrations in marine and freshwater, can also be excreted via the gills.

The flow-through system according to the invention comprises a number of fluid-permeable elements that operate according to the gill principle. In this context, the fluid-permeable elements can have a lamellar, comb-shaped, leaf-shaped, tuft-shaped or tree-shaped structure, so that the largest possible surface area can be created for gas exchange (O₂/CO₂). Preferably, the O₂ exchange to the fetal blood takes place via the countercurrent principle, which is greatly improved compared to conventional problem solutions. In this process, the modified amniotic fluid enriched with oxygen or with the oxygen-containing gas mixture is passed through the fluid-permeable elements of the flow-through system, while the fetal blood is passed by on the outside of the fluid-permeable elements.

Furthermore, also the CO₂-solubility in liquid (e.g. amniotic fluid) is about 24 times higher than that of oxygen with the gill structure according to the invention, whereas the passive diffusion in the tissue mainly depends on the diameter of the respective gas molecule. For example, oxygen O₂ with a molecular weight of 32 diffuses faster than CO₂ with a molecular weight of 44. As a result, marine animals such as fish, for example, are able to extract up to 90% of the available oxygen from the water with their gill system.

In one embodiment, the device according to the invention initially comprises a closed container which serves to accommodate the fetus as well as the flow-through system. When the device is used, the fetus is located in modified amniotic fluid and is supplied with oxygen or the oxygen-containing gas mixture and other vital substances via its umbilical cord. In the interior of the container at least one flow-through system is arranged, preferably two or more flow-through systems, which may run independently or in parallel. Each flow-through system consists of a larger number of fluid-permeable elements, for example >5, preferably >10 fluid-permeable elements, preferentially >50 fluid-permeable elements. These may, for example, consist of membranes, tubes or microporous material.

Connecting elements are provided for connection to the two umbilical arteries and the umbilical vein of a premature infant, connecting the blood vessels of the umbilical cord to the flow-through system via arterial catheters and venous catheters. This may be a single catheter with multiple lumens or multiple separate catheters. The flow-through system further comprises one or more flow-through lumens, which are provided for the passage of modified amniotic fluid.

Modified (or artificial) amniotic fluid relates to amniotic fluid that has been adapted to the fetus, with one or more components added or omitted. The components are, for example, electrolytes such as NaCl or KCl, or medicaments, nutrients or medical products.

Depending on the embodiment of the inventive device, amniotic fluid is passed from the outside in a longitudinal direction through the fluid-permeable elements of the flow-through system. In one embodiment, oxygen-enriched amniotic fluid (oxygenated amniotic fluid) is passed through the fluid-permeable elements. In another embodiment, fetal blood could be passed through the fluid-permeable elements. The amniotic fluid flows around the fluid-permeable elements, i.e. the membranes or capillaries, on the outside. Fixation of the blood vessels of the umbilical cord is achieved by a fixation system, for example a stent. For the introduction of amniotic fluid and/or breathing gas (oxygen, oxygen-gas mixture or Carbogen), at least one connection is provided on the container for conducting modified amniotic fluid and/or breathing gas into or to the flow-through system.

The gill-like design of the at least one flow-through system further enables the function of a dialysis system to dispose of substances. Preferably, in the method according to the invention, electrolyte exchange, disposal of toxins and waste products, in particular of bilirubin, ammonia, nitrogen, and osmoregulation are performed via the flow-through system.

In another embodiment, the modified amniotic fluid for the gill system is provided and used separately from the amniotic fluid of the artificial womb system in order to adjust fetal blood parameters and/or to provide fetal treatment via the gill system. Preferably, the modified amniotic fluid provided in a separate container optionally contains medicaments, heparin, vitamins, proteins, growth factors and/or hormones. It is particularly preferred that gas exchange and/or medicament administration and/or electrolyte administration and/or microelement administration and/or disposal of toxins and waste products, e.g. of bilirubin, ammonia, nitrogen, and/or plasma osmoregulation is provided via the separate amniotic fluid system. Alternatively, supply via the artificial womb system is also possible.

In another embodiment, the artificial gill system is located outside the artificial womb system to reduce noise exposure to the fetus. This may be within a further container or housing. However, in a preferred embodiment, the flow-through system is provided without a container at all, i.e, it is located outside the artificial womb and is not itself enclosed by a housing.

The device according to the invention is constructed like an artificial womb system so that the fetus develops in the modified amniotic fluid inside the container. The exchange between the fetal blood and the modified amniotic fluid takes place via the ultra-thin skin, the mucous membranes and the intestine of the fetus. Fetal oxygen demand is approximately 5 ml/min/kg (Campbell et al., J. Physiol 1966; 182:439-464). In the human fetus at 22 to 25 WG with a fetal weight of 300 to 500 g, the oxygen demand is about 2 to 4 ml/min. In this case, one ml of oxygen weighs the equivalent of about 1.34 mg. At an oxygen content in amniotic fluid of 7 to 50 mg/l, the fetal oxygen requirement can be almost completely covered at a water perfusion of 0.2 to 5 l/min, depending on the intrauterine pressure, number and structure of the fluid-permeable elements of the flow-through system (e.g. membrane thickness).

The fluid-permeable elements preferably include membranes, membranes with micropores (polymethylpentene; PMP material) or membranes with micropores. However, other fluid-permeable elements are also covered by the invention, which operate according to the gill principle and have gas-permeable channels for O₂ and CO₂. Furthermore, the flow-through system may also employ other fluid-permeable elements that provide transport channels or micropores for the detoxification function or for the normalization of electrolytes (e.g. Cl⁻ and Na⁺).

The flow-through system is connected to the infant's vascular system (i.e. via the infant's umbilical cord) via the connecting elements, preferably via a port system. In a first embodiment, the gassed amniotic fluid (e.g. provided as O₂ or O₂/CO₂ or O₂/CO₂ and nitrogen mixture) is pumped directly over the stacked fluid-permeable elements. Preferably, a pressurized container of amniotic fluid is provided for this purpose. Alternatively, a pump can be used to pass the amniotic fluid enriched with oxygen or the oxygen-containing gas mixture through the fluid-permeable elements. In a further embodiment, oxygen can also be supplied directly by gassing the interior of the container with oxygen or an oxygen-containing gas mixture. Appropriate connections for the supply of breathing gas are provided for this purpose. In this way, the individual proportions for oxygenation of the fetus can be precisely adjusted, in particular the ratio of amniotic fluid/gas, amniotic fluid/oxygen. These ratios can range from 0.1/10 to 9.9/10. The gas exchange is controlled by the velocity of the amniotic fluid flowing through the flow-through system, the fluid volume, the direction/opposite direction, the frequency (oscillation 0-1000 Hz), the O₂ supply and/or gas mixture supply and/or by a pressure change in the flow-through system. Preferably, the flow-through system comprises a pressure valve which is integrated at the umbilical outlet of the flow-through system. Preferably, this is a pressure flap that can be mechanically adjusted in advance. Opening and closing of the flap can take place mechanically and/or digitally. The countercurrent principle facilitates O₂ uptake from the amniotic fluid into the fetal blood, similar to a natural gill system.

In another embodiment, the fetal blood flows directly through the fluid-permeable elements, preferably via small tubes or a membrane system, where gas exchange occurs. The modified amniotic fluid (with or without oxygen) or an amniotic fluid/gas mixture (amniotic fluid/gas ratio of 0.1/10 to 9.9/10) is passed through the fluid-permeable elements and accelerated in flow rate, preferably by a tapering diameter of tubes. The pressure valve can be used to increase the water pressure in the system on the fluid-permeable elements (e.g. membranes). Herein, the pressure can be increased periodically or kept at a constant level. The pressure in the flow-through system can also be controlled by gassing the amniotic fluid. Preferably, the modified amniotic fluid in the flow-through system is oscillated via the pressure valve or via a supply device, preferably at a frequency of 0-1000 Hz.

It is also conceivable, for example, that the flow-through system (i.e. the gill system) could be used independently of the artificial womb system, for example, to replace or possibly supplement lung function in children or adults.

Depending on the embodiment, the modified amniotic fluid can also be replaced by other solutions or fluids, for example blood, plasma, nutritional solutions, saline solutions (NaCl), plasma replacement solutions, sea water, etc.). The flow-through system according to the invention preferably works according to the acceleration principle, in which the amniotic fluid (or other liquid) is accelerated by a pump, making the oxygen/CO₂ exchange more efficient.

In a preferred embodiment, the flow-through system of the device according to the invention additionally comprises an additional absorber to dispose of substances such as cytokines, toxins, ammonia, bilirubin, myoglobin, creatinine, inflammatory substances or degradation products from the fetal blood. The cytokines are, for example, IL-6, IL-8, IL-10, TNF-alfa, IFN. Preferably, the modified amniotic fluid is preheated to a temperature between 37° and 39° C. via a heating device. Preferably, the interior of the container (corresponding to an artificial womb) is also maintained at a temperature between 37° and 39° C. Temporary cooling to a temperature of up to about 34° C. can be provided via a thermoregulator to reduce organ damage following asphyxia. Preferably, the flow-through system further comprises a measuring device for measuring the oxygen saturation in the amniotic fluid of the artificial womb system.

For successful life support and further development of the premature infant, the composition of the modified amniotic fluid is also crucial. If the composition of the amniotic fluid is insufficient, there is a risk that fetal internal organs, umbilical cord, amnion, skin, eyes or mucous membranes will suffer irreversible damage.

In a preferred embodiment, the amniotic fluid used according to the invention therefore comprises a nutrient composition whose concentrations correspond to the physiological situation in the fetus at the respective gestational age. Accordingly, the concentration of fatty acids, vitamins, microelements, growth factors, hormones, electrolytes, cytokines and other regulatory substances is constantly monitored, adjusted and substituted as required. Preferably, the container of the artificial womb of the womb system according to the invention comprises modified amniotic fluid which is composed according to U.S. Pat. No. 9,072,755 B2.

Other trace elements, such as boron, chromium, iron, fluorine, iodine, cobalt, lithium, manganese, molybdenum, nickel, silicon, vanadium, amino acids, growth factors, vitamins and hormones can supplement the modified amniotic fluid. Preferably, the amniotic fluid is preheated to a temperature between 37° C. and 39° C. and simultaneously gassed with oxygen or an oxygen-containing gas mixture before being introduced into the artificial womb.

After cutting the umbilical cord, fetal O₂ saturation is maintained at 60-90% in the artificial womb system by the flow-through system in the gassed amniotic fluid.

The present invention further relates to an ex-vivo method for life support of a human being, preferably a newborn, in particular an extremely premature infant between the 21/0 and 28/0 WG for maintaining its vital functions. The features previously described for the device apply mutatis mutandis to the method. The method is applicable to children and adults. In the method, amniotic fluid enriched with oxygen or an oxygen-containing gas mixture or a plasma substitute solution is supplied to a container in which there is at least one flow-through system consisting of a number of fluid-permeable elements and connecting elements for connection to the catheters for the umbilical arteries and umbilical vein of a human (e.g. premature infant) and a flow-through lumen for passing modified amniotic fluid or the plasma replacement solution through the fluid permeable elements, and at least one connection for introducing oxygenated modified amniotic fluid, plasma replacement solution and/or breathing gas into the flow-through system. Thereby, either the amniotic fluid respectively plasma replacement solution or the blood is passed through the fluid-permeable elements. Preferably, the flow-through system maintains a pressure between 5 mbar and 5 bar.

Preferably, the premature infant is a fetus born before the completed 28th week of gestation. However, the womb system according to the invention and the ex-vivo method also function in children who have a limited life expectancy due to lung insufficiency, for example, caused by congenital defects or due to functions that have not yet been developed, as can be seen, for example, in newborns with hypoplasia of the lungs. With the aid of the device respectively method according to the invention, permanent treatment is possible of pulmonary insufficiency or damage to airways or lungs caused, for example, by burns.

In a preferred embodiment, it is provided that monitoring and regulation of the vital functions of the newborn (in particular the physiological fetal parameters) are carried out via a computer network using artificial intelligence, which also enables data exchange via the internet. Remote regulation of the system via a network using artificial intelligence is also possible. Via the computer network, data of vital signs such as concentrations of CO₂ or O₂, saturation, blood flow volume, blood values, amino acids, coagulation status, fatty acids, glucose, growth factors, the CRP and other inflammation parameters such as IL-6, pro-calcitonin, metalloproteinases and other cytokines will be monitored and continuously analyzed. Bacterial colonization will also be monitored and analyzed. The analysis is intended to provide information about the acute health status of the infant as well as to enable a steadily more precise risk prognosis. For this purpose, chips almost permanently send the corresponding values to an evaluation unit. The complete analysis of the data can be carried out via a central unit for each individual womb system. Parents can be integrated into fetal video monitoring or vital signs monitoring as needed or desired, e.g. via a smartphone. This type of parental integration also allows for audio and/or video-based communication, enabling interaction between the parents and the infant. This is especially important because mothers often suffer from fears of loss or failure. Audio and/or video-based interaction between mother and fetus, e.g. via a smart phone, is beneficial for preventing depression in the mother, among other things. For example, the voice of the parents, breathing, heartbeats, and even bowel sounds that are part of a natural environment can be transmitted.

In one embodiment, it is provided that, for example, amplified heartbeats of the child, after filtering out the device noises, are transmitted to the mother in real time via a communication device (e.g. smartphone). In turn, sounds of the mother and/or father (e.g. heartbeat, voice, breathing sounds, possibly bowel sounds) can be acoustically broadcast into the womb system in real time (“live”) or via a continuous loop.

According to the invention, facilities are provided that enable high-frequency data acquisition and analysis. In particular, the analysis includes the use of density prediction methods, appropriate risk prediction, and the implementation of regime switching models.

For example, a Markov-switching GARCH specification according to Haas et al. (2004), but with additional consideration of delayed cross-correlation between the blood value time series and the effect of exogenous interventions, could be used with the system or method of the invention to assign a certain probability to their different behavioral patterns (Haas M, Mittnik S, Paolella M S 2004; Hastie et al. 2008). Precise estimation of parameters and probabilities can be calculated, for example, via EM algorithms.

High observation cycles of certain patterns in regimen assignment, especially before the onset of a pathologic condition, could provide a tremendous gain in information for understanding a disease. For this pattern recognition, the use of an artificial neural network could be explored to estimate the likelihood of a need for medication coming up. Therefore, in a preferred embodiment, continuous analysis and estimation of neonatal outcome should be performed for correction of the therapy and adjustment of the setting in the flow-through system.

In a preferred embodiment, the device according to the invention comprises a communication system, which enables the transmission of audio signals between the fetus in the artificial womb system and the mother or father. Preferably, the device is provided with an audio system which enables communication between the fetus in the artificial womb system and the mother or father. In this way, for example, speech, tones or sounds can be transmitted from the outside to the inside of the womb system. Digital filters can be used to digitally filter out non-natural noises (e.g. gassing noises, noises from machines, devices, etc.). Furthermore, acoustic heart actions of the fetus are to be recorded and, if necessary, digitally amplified in order to transmit this information via a network to a smartphone of the mother and/or father for example.

In a further developed embodiment, the device according to the invention comprises more than one flow-through system, preferably two or more flow-through systems, which are connected either in parallel or in series. This makes it possible to run several systems in parallel or independently of each other. This is important, for example, when changing the oxygenator. Furthermore, in case of need, the existing redundancy allows a defective system to be replaced by a functioning system, in order to ensure, for example, O₂ gassing, an increase in pressure in the flow-through system or an increase in the flow of amniotic fluid, thus maintaining the physiological functions of the infant. These functions should also be networked in such a way that the systems can be monitored and, if necessary, controlled worldwide via a network. The recording, acquisition and transmission of fetal vital signs and other information, such as audio information, are preferably transmitted via an encrypted network. It should be possible to regulate the vital functions via control software and a control system integrated in the womb system (e.g. for gas exchange, amniotic fluid supply, supply of fetal blood). In particular, the control system controls the flow rate, the accumulation of O₂ in the amniotic fluid andior the concentration of breathing gas in the interior of the container, etc.

Multiple flow-through systems also have the advantage of reducing the risk of contamination and also enabling medication administration via the amniotic fluid of the artificial gill system. In addition, indirect electrolyte, osmolarity or plasma regulation of the fetus is possible via adjusting the formula of the modified amniotic fluid for the flow-through system as an artificial gill system.

The invention has significant advantages compared to conventional oxygenators. The inventor of the present invention has found that the devices and methods described in the prior art are inadequate for the sole reason that the use of an oxygenator in an artificial extrauterine system is associated with a high level of heparin substitution. A very high incidence of intracranial cerebral hemorrhage in extremely premature infants remains an unresolved problem in pediatrics (see Younge et al., New Eng J Med 2017 paper). High-dose heparin substitution significantly increases the risk and extent of cerebral hemorrhage in children. Additionally, current oxygenators deliver too much O₂ with a very large reduction in CO₂, which is not physiological for the fetal situation and is associated with complications. Stabilization of fetal blood gases was achieved by an additional complicated new mixture of gases used in oxygenators (O₂, CO₂ and N₂). Due to the function of the oxygenator, complications often occur, such as increased risk of thrombosis, destruction of erythrocytes, anemia and hyperbilirubinemia.

Furthermore, according to the invention, it is additionally or alternatively possible to convert known oxygenators to “moist” gassing (e.g. via modified amniotic fluid, plasma substitute solutions, electrolyte solutions, etc.) instead of O₂ gassing.

Replacing the gas mixture with perfusion with an O₂-enriched modified amniotic fluid reduces the risk of thrombosis and heparin requirements, contributes to the physiological stabilization of fetal blood gases, and increases the durability of the flow-through system.

### BRIEF DESCRIPTION OF THE DRAWINGS

The invention is explained in more detail in the following figures.

FIG. 1 shows a first embodiment of the device in which oxygenated modified amniotic fluid flows through membranes of the flow-through system (gill system),

FIG. 2 shows another embodiment n which the fetal blood flows through the flow-through system,

FIG. 3 shows an embodiment of an artificial womb system,

FIG. 4 shows another embodiment with two flow-through systems,

FIG. 5 shows an embodiment with a second amniotic fluid system for supplying the fetus with medicaments,

FIG. 6 shows the structure of an embodiment of the artificial gill system.

### WAYS OF CARRYING OUT THE INVENTION

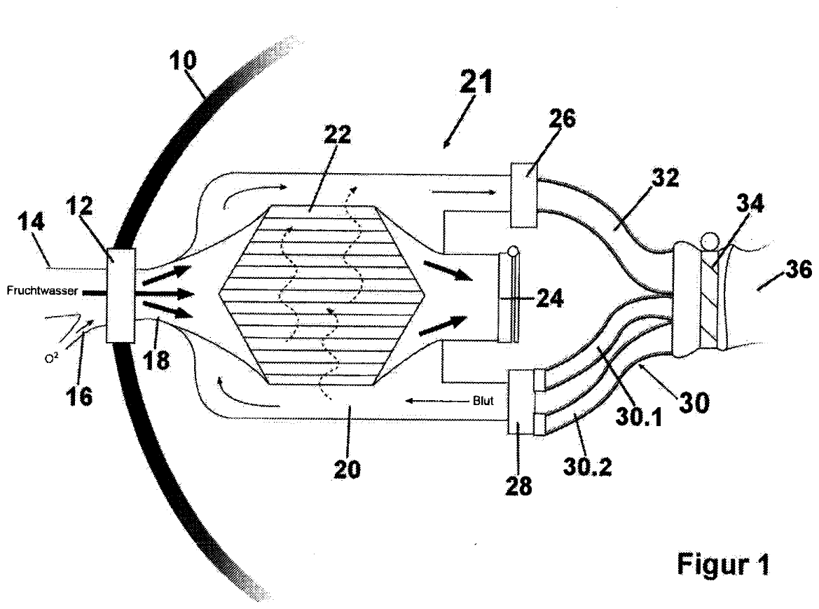

FIG. 1 shows the device according to the invention and its individual components. The device comprises a container 10 in which a flow-through system 21 is arranged. Via a pump system, modified amniotic fluid (preferably preheated) is introduced via a connection 12 into the flow-through system 21 through a line 14 via a connection piece 18. Oxygenation of the amniotic fluid may be provided via an oxygen line 16. Nevertheless, it is also provided that the artificial space inside the container can be gassed with medical breathing air via its own gas port. The flow-through system 21 itself comprises a number of more than 20 fluid-permeable elements which are permeable to O₂ and CO₂. Preferably, these are lamellar membranes or a number of tubes with integrated micropores for gas exchange. The catheter connection for the umbilical vein catheter 32 and the two umbilical artery catheters 30.1; 30.2 to the umbilical cord 36 of the fetus is made via connecting elements 26, 28 and at the other end via a port system 34. Via a pressure valve 24, the internal pressure of the gill system can be regulated at a frequency of 0-1000 Hz, preferably 10-80 Hz. Fetal blood is directed in accordance with the countercurrent principle via corresponding flow-through lumens 20 around the membrane of the flow-through system 21. This enables an efficient exchange of oxygen dissolved in the amniotic fluid and the fetal blood. By increasing the flow rate, the efficiency of the O₂/CO₂ exchange can be significantly enhanced. The pressure in the flow-through system is preferably maintained between 5 mbar and 5 bar. Inlets for oxygen or a gas mixture are provided. Furthermore, admission of a gas mixture of O₂, CO₂ and N₂ can also be provided. Alternatively or additionally, the Carbogen gas mixture or O₂ can also be admitted directly into the amniotic fluid.

The pressure valve 24 is used in addition to the amniotic fluid pump (not shown) to control the amount of amniotic fluid, the velocity and the pressure in the flow-through system 21. The control of the pressure valve 24 can be mechanical or digital. A sample for a measuring device can be taken via a measuring supply line (not shown), for example for determining the oxygen saturation in the blood before and after oxygenation.

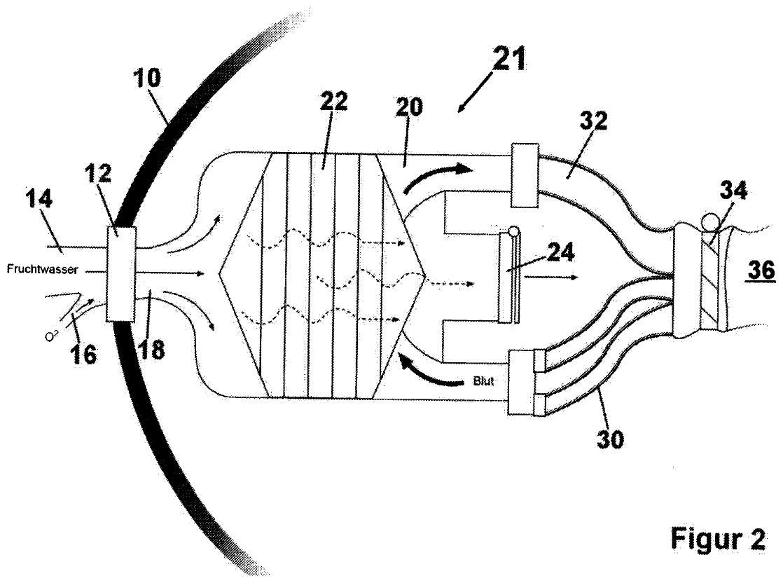

FIG. 2 shows another embodiment in which the fetal blood directly flows through the fluid-permeable elements 22 of the flow-through system 21. In this embodiment, the modified amniotic fluid flows around the fluid-permeable elements 22, i.e. the capillaries or membranes.

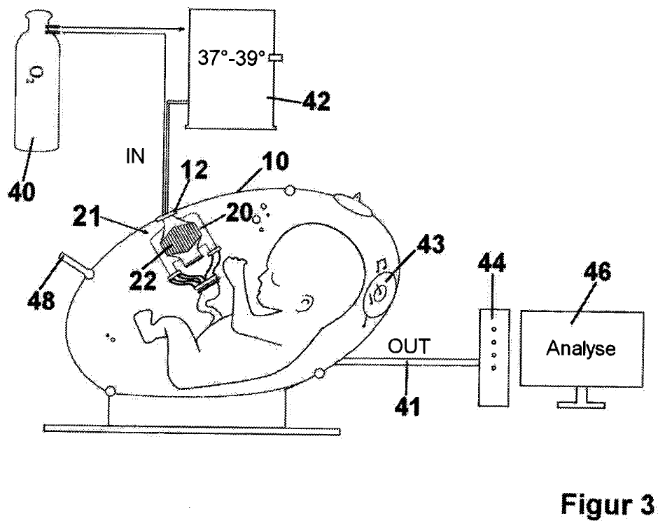

FIG. 3 shows an embodiment of the device according to the invention as a vital system. The container 10 comprises a flow-through system 21. A connection 12 is used to supply modified amniotic fluid, which is preferably provided in a preheated storage container 42. For this purpose, the storage container 42 is preferably equipped with a thermostat and a pump. Preferably, a preheating temperature of 37° to 39° C is aimed for in the storage container 42 for the modified amniotic fluid in the gill system. Oxygenation of the amniotic fluid may be provided directly from an oxygen tank 40. Alternatively or additionally, direct gassing with oxygen of the artificial space of the container 10 and/or the flow-through system 21 may be performed. Via corresponding sensors 48, it is possible to monitor vital functions and to forward them via a network. Furthermore, it is possible to record and play sounds and noises via an audio device 43. Data are transmitted via a network 41 to corresponding servers 44 and ultimately analyzed via an analysis device 46. In the same way, the entire system can also be regulated.

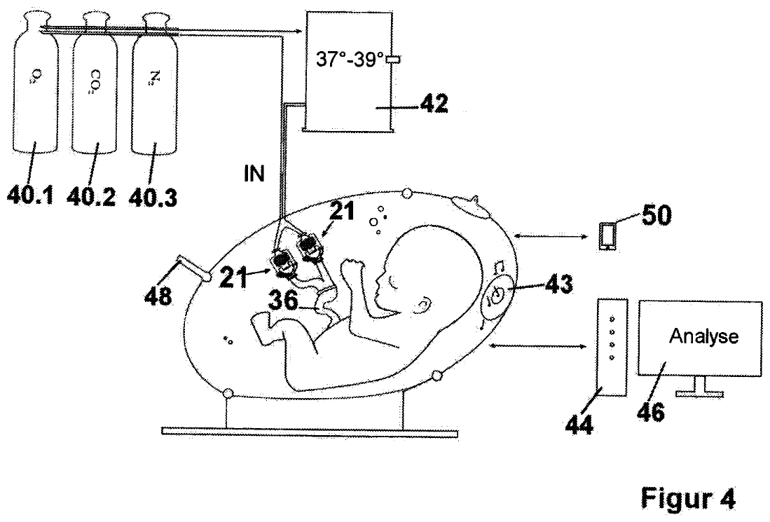

In FIG. 4, an analogous system is shown, which is constructed similarly to the embodiment of FIG. 3. Herein, the gassing is carried out with a gas mixture of O₂, CO₂, N₂, provided in tanks 40.1, 40.2, 40.3. The addition of CO₂ can be useful, for example, for the decompression of vessels. According to the invention, two flow-through systems 21 are provided. However, as with the other embodiments, data exchange can also be performed via a smartphone 50, i.e. important vital signs, audio files or other information from and to the artificial womb system can be exchanged directly by the mother/father or other person monitoring the womb system.

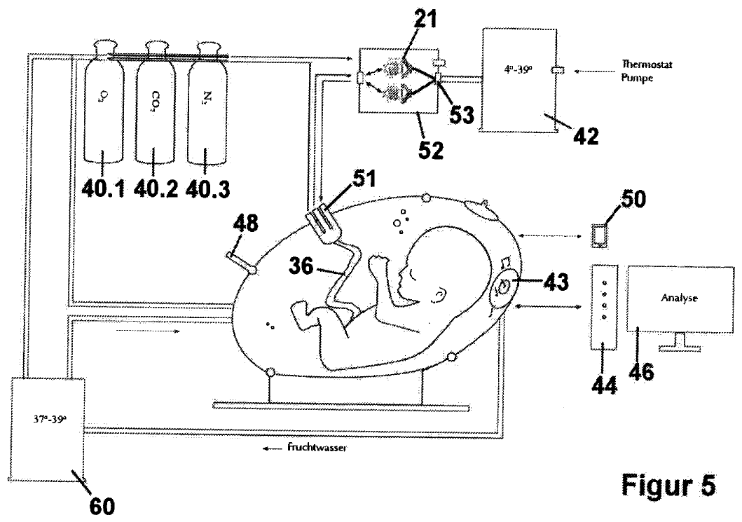

In FIG. 5, a more advanced system is shown in which an additional container 60 with modified amniotic fluid is provided next to the storage container 42 filled with modified amniotic fluid. The fetus is supplied with the substances via the flow-through system 21, i.e. via artificial amniotic fluid enriched with the substances, which is provided by the storage container 42. This facilitates supplying the fetus with medicaments, including heparin, vitamins, proteins, growth factors and/or hormones. Via administering modified amniotic fluid to the fetus, which is supplied by the artificial gill system, for example neonatal care can be provided or prophylactic or therapeutic treatment can be carried out. For example, blood parameters of the fetus can be adjusted individually.

Preferably, water-soluble substances are used, which dissolve in the modified amniotic fluid. The system has the advantage that overhydration of the premature infant is avoided, because it prevents an increase in volume due to the absorption of too much fluid. Furthermore, decompensation occurring with other systems or treatment methods is prevented, because at least some of the active and nutritional substances can be absorbed via the gill system, thus avoiding unnecessary volume loading of the fetus. In this embodiment, it is therefore provided that the modified amniotic fluid of the storage container 42 for the gill system is separate from the amniotic fluid of the uterine system in the container 60. For example, the administration of heparin or other antithrombotic medicaments can be administered locally via the artificial gill system (flow-through system 21) to have an antithrombotic effect. Systemic exposure of the fetus to heparin is avoided.

In this embodiment, it is further provided that the flow-through system 21 is arranged in an external housing 52 as an external gill system. The umbilical cord 36 of the fetus is connected to the external artificial gill system via a port system 51. For optimal oxygenation, the temperature of the modified amniotic fluid in the storage container 42 can be cooled down to as low as 4° C. to increase the oxygenation of the amniotic fluid several times. Temperatures in the range of 4° C. to room temperature (about 21° C.) are preferred. However, temperatures as high as 39° C. are also possible. The admission of modified amniotic fluid from the storage container 42 to the flow-through system 21 is controlled by a valve 53.

In another embodiment (not shown), the storage container 42 for modified amniotic fluid equipped with a thermostat is omitted. In this embodiment, the function of the storage container 42 equipped with a thermostat and a pump is integrated into a housing 52. Accordingly, the housing 52 may include a pump and a thermostat.

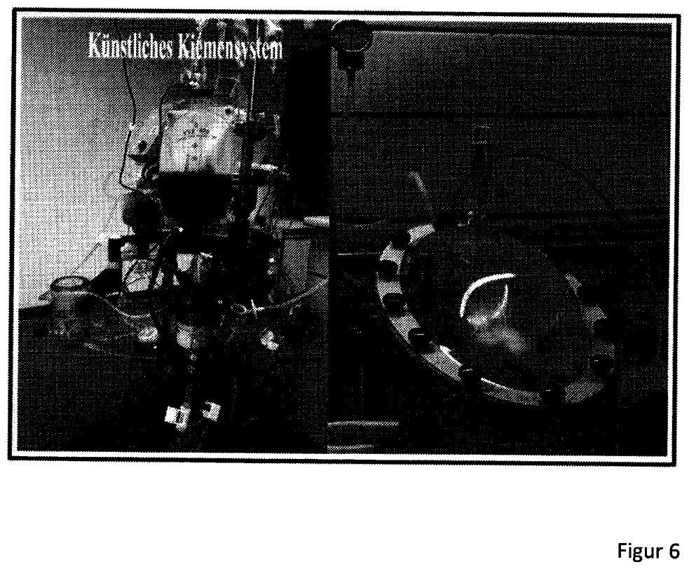

FIG. 6 shows a ready-to-use device constructed according to the specifications of the invention.

1. A device for the life support of a newborn, in particular of an extremely premature infant between the 21/0 and 28/0 weeks of gestation (WG), comprising at least one flow-through system (21) comprising a number of fluid-permeable elements (22) and connecting elements (26, 28) configured for connection to the umbilical artery catheter (30) and umbilical vein catheter (32) of the newborn, and a flow-through lumen (20) for passing modified amniotic fluid enriched with oxygen or an oxygen-containing gas mixture through the fluid-permeable elements (22), and at least one connection (12) for introducing the oxygen- or oxygen-containing gas mixture-enriched modified amniotic fluid into the flow-through system (21), wherein the flow-through system (21) is configured to pass the oxygen- or oxygen-containing gas mixture-enriched modified amniotic fluid through the fluid-permeable elements (22) of the flow-through system (21) while passing the fetal blood past the outside of the fluid-permeable elements (22) via the flow-through lumen (20), whereby gas exchange takes place.

2. The device according to claim 1, characterized in that the flow-through system (21) is arranged in a container (10).

3. The device according to claim 1, characterized in that oscillation and pressure changes of the modified amniotic fluid in the flow-through system (21) are effected via a pressure valve (24) or via a supply device, preferably at a frequency of 0-100 Hz.

4. The device according to claim 1, characterized in that the fluid-permeable elements (22) of the flow-through system (21) are arranged or structured in a gill-like manner.

5. The device according to claim 4, characterized in that the fluid-permeable elements (22) have a lamellar, comb-shaped, leaf-shaped, tuft-shaped or tree-shaped structure.

6. The device according to claim 1, characterized in that the arrangement of the fluid-permeable elements (22) and the flow-through lumen (20) is such that the modified amniotic fluid enriched with oxygen or an oxygen-containing gas mixture flows through the fluid-permeable elements (22).

7. The device according to claim 1, characterized in that the fluid-permeable elements (22) are a plurality of membranes, tubes or microporous material arranged in series.

8. The device according to claim 1, characterized in that an additional absorber is provided for the disposal of cytokines, toxins, ammonia, bilirubin, myoglobin, creatinine, inflammatory substances or degradation products from the fetal blood.

9. The device according to claim 1, characterized in that devices are configured to facilitate monitoring of the vital functions of the newborn or regulation of the physiological fetal parameters via a network using artificial intelligence.

10. The device according to claim 1, characterized in that a housing (52) is equipped with a pump and a thermostat.

11. The device according to claim 1, characterized in that a pump for introducing the modified amniotic fluid is arranged at the flow-through system (21).

12. The device according to claim 1, characterized in that a storage container (42) is provided, via which medicaments, nutrients or medical products are additionally introduced into the flow-through system (21).

13. The device according to claim 1, characterized in that it comprises two or more flow-through systems (21).

14. The device according to claim 1, characterized in that it comprises a communication system configured to provide interaction between the fetus and the mother or the father via transmitting audio and/or video signals, in particular transmitting the parental voice, breathing, heartbeats or bowel sounds.

15. An ex-vivo method for life support of a human being, in particular extremely premature infant between the 21/0 and 28/0 weeks of gestation (WG), in which modified amniotic fluid or a plasma substitute solution is first enriched with oxygen or an oxygen-containing gas mixture and the enriched amniotic fluid or the plasma substitute solution is supplied to a flow-through system which consists of a number of fluid-permeable elements and connecting elements configured for connection to the arteries and veins of the human being and a flow-through lumen for passing the modified amniotic fluid through the fluid-permeable elements, and at least one connection for introducing the modified amniotic fluid enriched with oxygen or an oxygen-containing gas mixture into the flow-through system, wherein the oxygen-enriched modified amniotic fluid is passed through the fluid-permeable elements of the flow-through system while the blood is passed on the outside of the fluid-permeable elements via the flow-through lumen, and whereby gas exchange takes place.

16. The method according to claim 15, characterized in that there is a digital real-time connection between the fetus and the parents, in which the fetus is supplied with sounds of the mother and/or the father, preferably the voice, breathing, heartbeats and possibly bowel sounds.

17. The method according to claim 15, characterized in that electrolyte exchange, toxin and waste product disposal, in particular of bilirubin, ammonia, nitrogen, and osmoregulation are performed via the flow-through system.

18. The method according to claim 15, characterized in that, alternatively or additionally, moist gassing is carried out via modified amniotic fluid, plasma substitute solutions, electrolyte solutions.

19. The method according to claim 15, characterized in that the gas exchange in the flow-through system is controlled by the velocity of the amniotic fluid flowing through the flow-through system, the fluid volume, the direction/opposite direction, the frequency (oscillation 0-1000 Hz), the O₂ supply and/or gas mixture supply and/or by a pressure change in the flow-through system.

20. The method according to claim 15, characterized in that the amniotic fluid is preheated to a temperature between 37 and 39° C. and simultaneously gassed with oxygen or the oxygen-containing gas mixture before being introduced into the at least one flow-through system,

21. The method according to claim 15, characterized in that a pressure between 5 mbar and 5 bar is maintained in the flow-through system.

22. The method according to claim 15, characterized in that medicaments, nutrients or medical products are additionally introduced into the flow-through system.

23. The method according to claim 15, characterized in that the flow-through system is arranged either inside or outside a container.
